(19) United States
(12) Patent Application Publication
(10) Pub. No.: US 2019/0200865 A1
(43) Pub. Date: Jul. 4, 2019

(54) SYSTEM AND METHOD FOR ANALYZING BIOCHEMICAL SENSOR DATA

(71) Applicant: Profusa, Inc., South San Francisco, CA (US)

(72) Inventors: Ben HWANG, Lafayette, CA (US); William A. MCMILLAN, La Honda, CA (US); Natalie WISNIEWSKI, San Francisco, CA (US)

(73) Assignee: Profusa, Inc., South San Francisco, CA (US)

(21) Appl. No.: 16/235,245

(22) Filed: Dec. 28, 2018

Related U.S. Application Data
(60) Provisional application No. 62/611,184, filed on Dec. 28, 2017.

(51) Int. Cl. A61B 5/00; A61B 5/01; A61B 5/0205

(52) U.S. Cl. CPC A61B 5/0031; A61B 5/486; A61B 5/4866; A61B 5/7282; A61B 5/024; A61B 5/01; A61B 5/0205; A61B 5/6873; A61B 5/6878; A61B 5/445

(57) ABSTRACT

A system includes implantable sensors and monitors and are operable to provide health-related feedback to users based on data received from the implantable sensors and monitors.

### CROSS REFERENCE TO RELATED APPLICATIONS

This application claims the benefit of provisional U.S. Patent Application No. 62/611,184, filed Dec. 28, 2017, the disclosure of which is hereby incorporated by reference in its entirety.

### FIELD

The present disclosure is in the field of biochemical sensors, and systems operable to combine data from implantable sensors with data from optionally external monitoring devices to provide health-related feedback.

### BACKGROUND

For the management of many conditions, the regular measurement of analytes in vivo is desirable. It has been a long-standing objective of both medical science and the military to implant sensors inside the human body that continuously and accurately determine a quantity, concentration and/or changes in physiologic, metabolic, or fatigue status; measure the concentration of biothreat or therapeutic agents in vivo; and/or provide early detection of disease prior to the onset of symptoms. It has long been desired that such sensors and/or measurements be non-invasive and involve minimal user maintenance. Furthermore, it is desirable to achieve sensor longevity of months to years in actual user environments.

For certain conditions, biochemical data from the sensor is sufficient for the user and/or a clinician to determine if an intervention is needed. For other conditions, additional data related to the user and/or the environment surrounding the user may be needed and/or helpful in evaluating whether intervention is advisable. Therefore, there is a need for the ability to combine in vivo biochemical data with other data sources that provide other information about the user and/or the environment surrounding the user.

Traditional external analyte measuring devices suffer from a number of drawbacks impeding their use in providing health-related feedback to users. For example, fingertip-type pulse oximeters can provide some insight into the measure of a user's blood oxygenation, but such measures may produce inaccurate readings in cold conditions, on hypertensive users, and in the presence of motion (e.g., exercise, shivering, tremors, etc.). Such deficiencies may be compounded when combined with data from othering monitoring devices to prepare health-related feedback that is based on multiple sources of data. A need therefore exists for improved systems and methods for analyzing biochemical sensor data.

### BRIEF DESCRIPTION OF THE DRAWINGS

FIG. 1 is a chart showing an example of a concentration of oxygen as measured by an implantable sensor disposed in a user's forearm and as measured by a fingertip-type pulse oximeter.

FIG. 2 is a schematic illustration of a system including a sensor, a reader, an analyzer, a monitor, and a data processing system, according to an embodiment

### DETAILED DESCRIPTION

Described herein are systems that typically include one or more implantable sensors, one or more monitoring devices providing information about the condition of the user or the user's environment, and one or more data processing systems that receive and analyze data from the one or more implantable sensors and data from the one or more monitoring devices.

There is a need for the ability to provide clinical guidance to a patient. Some embodiments described herein include one or more implantable sensors. As described in further detail herein, implantable sensors typically provide more accurate and/or more reliable data than traditional external sensors, and therefore facilitate the ability to provide new types of health-related guidance to patients. Similarly stated, systems described herein that include at least one implantable sensor can be used to perform health-related analyses and generate health-related recommendations that would not be possible without the use of an implantable sensor.

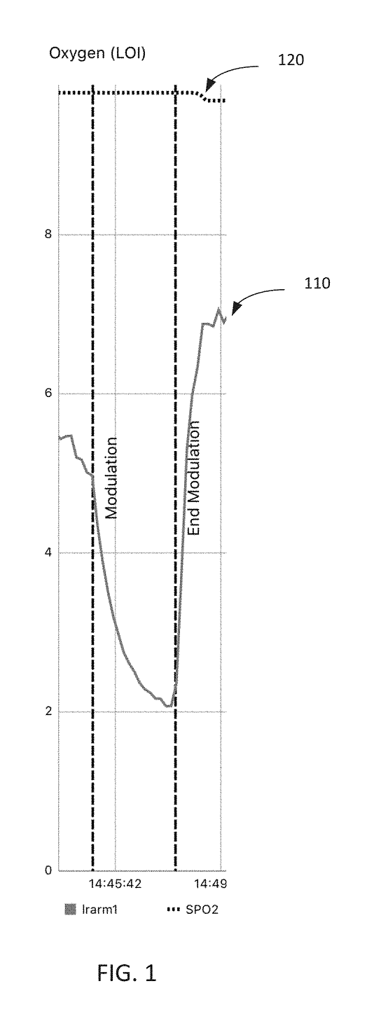

For example, known pulse oximeters operable to measure peripheral capillary oxygen saturation (SpO₂) suffer from a number of major drawbacks that generally render them unsuitable for use in systems described herein. In particular, known pulse oximeters are very sensitive to movement and are therefore generally unsuitable for continuously monitoring oxygen concentrations of active (e.g., exercising) users. Additionally, SpO₂ is generally an inferior measurement as compared to tissue oxygenation. FIG. 1 is a chart showing example data of tissue oxygen concentration as measured by an implantable sensor disposed in a user's forearm 110 and as measured by a fingertip-type pulse oximeter 120. When blood flow is modulated, in this example by inflating a blood pressure cuff, the implantable sensor detects a rapid decrease in tissue oxygenation concentration. The pulse oximeter, however, does not detect a decreased 402. Therefore, in an instance in which blood flow is interrupted known pulse oximeters may be unable to provide actionable data. For example as shown in FIG. 1, when blood flow is impeded a pulse oximeter may not be operable to detect the event, which can have catastrophic consequences if the impediment is associated with a clot or other medical emergency. In addition, in low-pressure and/or high-altitude conditions, a pulse oximeter may be affected by environmental conditions and report misleadingly low peripheral capillary oxygen saturation, while an implanted sensor will continue to report accurate tissue oxygenation. As yet another example, capillary constriction associated with cold temperatures and/or shivering can also effect 402 measurements taken by pulse oximeters such that measured 402 diverges from the more clinically useful measure of tissue oxygenation.

Some embodiments described herein relate to implantable sensors configured to measure glucose, lactose, lactase, oxygen, pyruvate, and/or any other suitable analyte. In some instances, such implantable sensors can be operable to measure a local concentration of an analyte. For example, as discussed in further detail herein, in some instances, an implantable sensor can supply analyte information associated with local wound healing or exertion of a particular muscle group that may not be available using known sensing techniques that detect a systemic concentration of the analyte (e.g., such as analytes measurements taken from a blood draw, finger stick, etc.) or a measurement of the analyte at a peripheral location (e.g., a fingertip, toe, etc.).

Some embodiments described herein relate to providing clinical guidance and include, involve, and/or make use of: (1) clinical grade data from implantable sensors and (2) data from other monitoring devices to assess other conditions of the user. In addition, some embodiments described herein include (3) analyzing clinical grade data from implantable sensors and data from other monitoring device and (4) providing guidance to the user and/or clinician as needed. Further, it may be desirable for (5) the clinician to have the ability to view all, some, or portions of the data, and send a notification to a user when a certain condition is met. In some embodiments, (6) the user may have the ability to view all, some, or portions of the data as well. Provided herein are methods and systems to achieve these goals. In some embodiments, a confidence level of the data can be determined and used, at least in part, to determine the appropriate guidance to provide to a user.

In an embodiment, the clinical grade data may be comparable to data from other clinically relevant sources.

In an embodiment, a system is described that includes: (1) one or more sensors (e.g., implanted and/or implantable sensors) that produce a signal in response to an analyte; (2) a reader that detects the signal from the one or more implantable sensors; (3) an analyzer that processes the signal from the reader and determines the amount or threshold presence or trend of the analyte detected by the one or more external sensors; (4) external monitors that provide information about a condition of the user or the local environment; and (5) data processing systems that receive data from the analyzer and data from the one or more external monitors, and processes the data to determine a condition of the user.

Some embodiments described herein relate to a system that includes an implantable sensor that is configured to produce a signal associated with a concentration of an analyte in a tissue (e.g., oxygen, glucose, lactate, carbon dioxide, H⁺, OH⁻, beta hydroxybutyric acid, cortisol, sodium, potassium, chloride, creatinine, urea, bilirubin, and the like.) Similarly stated, the implantable sensor can produce a signal (e.g., an optical signal) that is indicative of a level of the analyte in the particular tissue (e.g., subcutaneous tissue, muscle, stomach or intestinal tissue, pancreatic tissue, brain tissue, etc.). In some instances the implantable sensor therefore produces a signal indicative of a local concentration of the analyte. In some such instances, the implantable sensor can provide a location-specific measure of the analyte. In some embodiments, the system can include multiple implantable sensors configured to measure the same or different analytes. In some embodiments, an implantable sensor can be configured to produce multiple signals associated with concentrations of multiple analytes.

The system can also include a reader configured to be positioned on a surface of the user's skin above the implant. The reader can be configured to detect the signal emitted from the implantable sensor. For example, the reader can include a suitable optical sensor for detecting an optical signal originating from the implantable sensor and emitted through the skin of the user. In some embodiments, the reader can also be operable to calculate a concentration of the analyte based on the signal produced by the implantable sensor.

The system can also include a monitor configured to produce a signal associated with at least one of a condition of the user or a condition of an environment. Examples of monitors are discussed in further detail herein, but are typically external devices that may be coupled to the user or otherwise disposed in the vicinity of the user (e.g., within 100 feet) that detect a position of the user (e.g., geolocation, limb position, etc.), a proxy for an activity level of the user (e.g., heart rate, respiration, etc.), body temperature, ambient temperature, altitude, etc.

In some embodiments, the system also includes an analyzer communicatively coupled to the reader and the monitor. The analyzer can be operable to evaluate signals from each of the monitor and the reader and provide health-related feedback to the user based on the concentration of the analyte, the condition of the user, and/or the condition of the user's environment. In other embodiments, a device can perform the functions of one or more of the reader, the monitor, and/or the analyzer. Similarly stated, the analyzer, the monitor, and/or the reader may not be three distinct devices, but may be packaged in one or two housings.

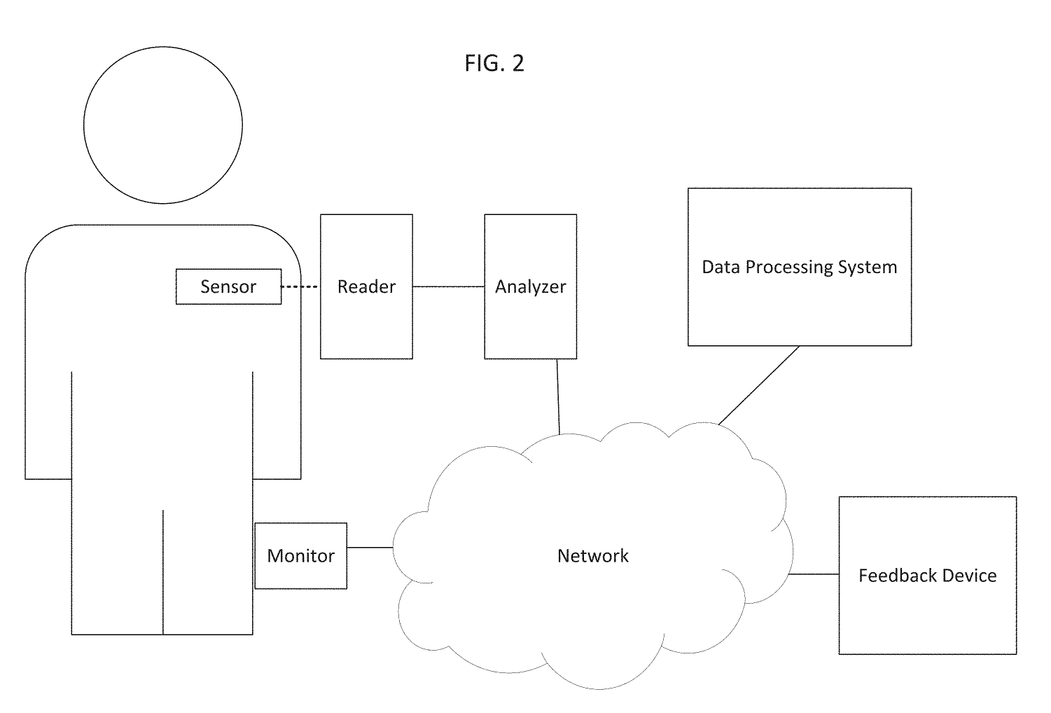

FIG. 2 is a schematic illustration of a system including a sensor, a reader, an analyzer, a monitor, and a data processing system, according to an embodiment. FIG. 2 also depicts a monitoring, information output, and/or feedback device. As shown in FIG. 2, the monitor, the analyzer, the data processing system, and the monitoring, information output, and/or feedback device are communicatively coupled by a network. In other embodiments, any other suitable communications connections between devices can be present. For example, the monitoring, information output, and/or feedback device may be directly communicatively coupled to the data processing system, the monitor may be directly communicatively coupled to the analyzer, and so forth.

It should be understood that although a reader, a monitor, and analyzer, a data processing system, and feedback devices are shown and described as discrete devices for ease of describing particular functions, it should be understood that any of these devices can be combined. For example, a “reader” can, in some embodiments perform the functions described below as being performed by a monitor, analyzer, data processing system and/or feedback device.

### A. Sensors

The sensors described herein may be implantable. Implantable sensors useful in the described system include tissue-integrating and non-tissue-integrating sensors. The implantable sensors may be implanted in a human or animal subject. Implantable sensors can measure an analyte, such as: oxygen, glucose, lactate, carbon dioxide, H⁺, OH⁻, beta hydroxybutyric acid, cortisol, sodium, potassium, chloride, creatinine, urea, bilirubin, and the like. The implantable sensors may produce an optically-detectable signal in the presence of the analyte.

In an embodiment, the implantable sensors may be tissue-integrating. Exemplary, non-limiting tissue-integrating sensors are described in published US patent application publication number 20120265034, filed Oct. 6, 2011, the contents of which are hereby incorporated herein in its entirety.

In an embodiment, the implantable sensors may produce a signal in response to an analyte. In an aspect, the signal may be an optical signal. The signal may be of sufficiently high quality to support medical-grade decision making. Similarly stated, the implantable sensor can produce clinical-grade data.

In a further embodiment, the implantable sensors may measure the amount of an analyte or the rate of change of the amount of an analyte. The data indicating the presence or amount of analyte may be of sufficient quality for clinical-grade decisions to be made.

### B. Reader

The reader detects the signal from the one or more implantable sensors. In an embodiment, the reader may be located outside the body. For example, the signal from the sensor can be an optical signal. The reader can include an optical assembly operable to receive a signal from the implantable sensor that is emitted through the tissue and, optionally, a light source to illuminate the implantable sensor. For example, the implantable sensor can be configured to be excited by an optical signal emitted from the reader and emit an optical signal (e.g., fluorescence, luminescence, etc.) associated with a concentration of the analyte in response to being excited.

The reader can be configured to be disposed on a surface of the user's skin above or near the reader. In some embodiments, the reader can include a processor and a memory and be configured to calculate a concentration of the analyte based on the signal from the implantable sensor. Exemplary readers are described in U.S. patent application publication nos. 2014/0275869, 2014/0364707, and 2016/0374556, which are hereby incorporated by reference herein in their entireties. Other readers are contemplated, as understood by one of ordinary skill in the art, for example, a reader can be configured to detect a radio or other non-optical signal from an implantable sensor.

In a further embodiment, the reader may be located inside the body.

### C. Analyzer

In an embodiment, an analyzer is provided that processes the signal from the reader and determines the amount or threshold presence or trend of the analyte detected by the one or more tissue-integrating sensors. In an aspect, the analyzer may be co-located with the reader. The analyzer may be in the same device as the reader. In a further aspect, the analyzer may be physically separate from the reader. In an aspect, the reader may transmit information to the analyzer. The information may be transmitted, for example, by wireless or wired means.

In an aspect, the analyzer may include a processor and memory storing software for processing the information received from the reader.

In an aspect, the analyzer may be co-located with the data processing system. The analyzer may be located in the same device as the data processing system. In a further aspect, the analyzer may be physically separate from the data processing system. In an aspect, the analyzer may transmit information to the data processing system. The information may be transmitted, for example, by wireless or wired means.

In an embodiment, the reader, analyzer, and data processing system may be located in the same device. In an aspect, the reader, analyzer, other monitoring device(s), and the data processing system are located in the same device.

### D. Monitor

In an embodiment, one or more monitors or monitoring devices may monitor a condition related to the user and/or the user's environment. The condition may or may not be associated with a disease or impaired health. In an embodiment, the monitor may a non-analyte sensor. In a further embodiment, the monitors may measure an analyte. In an embodiment, the monitor(s) may be co-located with the first sensor or may be part of the same sensor. The monitor may be external, non-implantable, and/or non-tissue integrating.

In an embodiment, the monitor(s) may be external to the subject. In an embodiment, the external sensor may be physically contacting the subject. In a further embodiment, the monitor(s) may not be in physical contact with the subject. The monitor(s) may be wearable or may not be wearable. Exemplary non-limiting monitors include: FitBit, mobile phone, heart rate monitors, blood pressure monitor, etc.

Contemplated monitors may provide information about one or more parameters related to the subject or of the subject's environment. Exemplary non-limiting parameters related to the subject that can be measured by the monitor(s) include: heart rate, activity level, galvanic skin response, blood perfusion, blood pressure, breathing rate, blood analytes, tissue analytes, and sweat, motion, gait type, geolocation, internal temperature of the subject, mood, heart rate variability, breathing pattern, body position, body part position, acceleration, velocity, and socialability (e.g. how many times they use a phone, and app, call someone). Exemplary non-limiting conditions of the user's environment that can be measured by the monitor(s) include: date/time, altitude, pollen count, smog index, pollution count, weather conditions, external temperature, and humidity.

### E. Data Processing Systems

Data processing systems described herein receive and analyze data from the one or more implantable sensors and data from the one or more external sensors.

In an embodiment, data from the implantable sensors may be communicated to an external device on or near the user. In an aspect, the data from the implantable sensors may be communicated to a computer or server that may or may not be located near the user. The data from the implantable sensors may be communicated to a distributed computing environment (e.g., “the cloud”).

In an embodiment, data from the monitoring device may be communicated to an external device on or near the user. In an aspect, the data from the monitoring device may be communicated to a computer or server that may or may not be located near the user. The data from the monitoring device may be communicated to the cloud.

In an embodiment, data from the implantable sensors and data from the monitoring device may be analyzed together and processed to determine which information to provide to the user (or clinician or caregiver).

Algorithms may be used to analyze the data and determine which information to provide to the user (or clinician or caregiver). Exemplary algorithms include linear relationship, non-linear relationship, machine learning algorithms, support vector machine, and/or neural network.

### D. Device Providing Information to a Subject or a Healthcare Provider

D1. Types of Devices

Devices that provide information to a subject or a healthcare provider are contemplated. Provided information will be dependent on the analysis by the data processing system.

D2. Types of Information Provided by Device

In an embodiment, information may be selected from one of more of the following: recommended lifestyle change, recommended intervention, medical diagnosis, medical prognosis.

In an embodiment, the non-tissue-integrating monitor may provide an indicator of the stress level of a user. In an embodiment, the stress indicator may be heart rate, cortisol, voice, noise, light, and/or galvanic skin response or the like.

In an embodiment, the non-tissue-integrating monitor may provide an indicator of dehydration and/or kidney function. In an embodiment, the dehydration and/or kidney function monitor may be selected from the group consisting of: urea, creatinine, potassium, sodium, activity level and temperature.

### EXAMPLES

### Example 1

Oxygen Sensors: Measuring Oxygen and Altitude

An oxygen sensor can be implanted in a user and oxygen levels can be detected using an external reader. An analyzer processes the signal from the reader and determines the amount or threshold presence or trend of the oxygen detected by the sensor. The reader and the analyzer may be located in the same device, or they may be located in different devices. The reader may send the oxygen data to an external server. The reader may send the oxygen data to a different device, which may or may not be located on the user.

An additional monitor determines geolocation and motion (as a measure of activity). The monitor may also measure breathing rate and/or heart rate or other biometrics. The geolocation and/or motion monitor may send the geolocation and motion data to an external server. The geolocation and/or motion monitor may send the geolocation and motion data to the sensor reader. The geolocation and/or motion monitor may send the geolocation and motion data to a different device, which may or may not be located on the user.

Oxygen data and the geolocation and motion data can be analyzed by the sensor reader, an external server, and/or any other suitable device. As shown in the table below, based on oxygen level, altitude measured, and activity level measured, feedback can be provided to the user.

| | | | Activity level measured | |
|---|---|---|---|---|
| | Oxygen level | | (measured by motion, | |
| | measured by | Altitude | breathing rate, and/or | Exemplary Feedback |
| | sensor | measured | heart rate) | to user |
| Combination 1 | low | high | high | Rest or use |
| | | | | supplemental oxygen |
| Combination 2 | low | high | Low or none | Move to lower altitude, |
| | | | | use supplemental oxygen, |
| | | | | or lower head relative |
| | | | | to heart |
| Combination 3 | low | low | High | Rest |
| Combination 4 | low | low | Low or none | Use supplemental oxygen |
| | | | | (bronchodilators or other |
| | | | | therapeutics) or seek |
| | | | | medical consultation |

### Example 2

Remote Monitoring of COPD Patients

Patients with COPD can be monitored remotely. Oxygen sensors can be implanted in the patients. In addition, the patients may also be implanted with one or more of: carbon dioxide sensors, and/or pH sensors. In addition, the patient may also have a bicarbonate sensor. The patient may also be wearing a movement and/or motion monitor. The patient may also be wearing a heat flux monitor. A clock in the sensor reader system, external monitoring device(s), or a connected smartphone will define time of day for events or change in state detection. A breathing rate/respiration rate monitor may also be used to monitor the breathing rate of a patient. The combination of wearable data combined with the chemistry data from the sensors can allow for interpretation of the condition of the patient.

For example, hypoxia and/or hypercapnia in the absence of motion at night may indicate that the patent did not use evening inhalers, the oxygen cannula came out of the nasal passage, or oxygen flow is otherwise disturbed. The user might receive a signal to: “Check medications or oxygen cannula or oxygen tank or tank and cannula connectors”. These same conditions occurring during the day, but after a known event showing the patient arose from bed in the morning, may alert a loss of consciousness.

For example, if hypoxia and/or hypercapnia were detected concurrent with vigorous exercise, the user might receive a signal to: “Please stop and rest”.

| | Oxygen level | Carbon dioxide | | |
|---|---|---|---|---|
| | measured by | measured by | Activity level measured | Exemplary Feedback |
| | sensor | sensor | (measured by motion) | to user |
| Combination 1 | high | low | high | No feedback needed |
| Combination 2 | low | High or low | high | Stop and rest; check |
| | | | | medications |
| Combination 3 | low | High or low | Low or none | Check medications; |
| | | | | take supplemental |
| | | | | oxygen; or consider |
| | | | | seeking medical care |

### Example 3

Remote Monitoring of PAD/CLI Patients

Patients with PAD/CLI can be monitored remotely to monitor the vascular perfusion status in PAD/CLI patients. Oxygen sensors will be implanted in the patients. In addition, the patients will be wearing two or more Inertial Measurement Units (IMUs). An IMU can be placed on the foot and another IMU can be placed on the abdomen or chest region, which can be used to determine the position of the patient's foot relative to the body position. Patient may also input particular challenges to the foot (e.g., a leg lift or pressure cuff) to help interpret the data. Another stream of data may include blood perfusion (e.g., laser Doppler, laser speckle) to help interpret blood flow in the limb and its relationship to oxygen concentration

For example, streaming data may be sent to the cloud to determine whether changes in vessel patency has occurred. Changes in patency results in changes in response to position/postural provocations. These measurements can be taken periodically to establish a trend. Health-related feedback can be provided to the user based on the oxygen level measured by the sensor, the torso position, and/or the foot position.

| | Oxygen | Torso | Foot | |
|---|---|---|---|---|
| | level | position | position | Exemplary |
| | measured by | measured by | measured by | Feedback |
| | sensor | IMU | IMU | to user |
| State 1 | Oxygen level | Patient | Foot on same | |
| | measured by | lying down | plane as | |
| | sensor | | torso | |
| State 2 | Oxygen level | Patient | Foot above | Oxygen level |
| | measured by | lying down | plane of | difference or |
| | sensor | | torso | rate of change |
| | | | | between state |
| | | | | 1 and state 2 |
| | | | | indicates level |
| | | | | of occlusion. If |
| | | | | occlusion |
| | | | | increases, alert |
| | | | | doctor. |
| State 3 | Oxygen level | Patient | Foot on | Oxygen level |
| | measured by | standing up | ground | difference or |
| | sensor | | | rate of change |
| | | | | between state |
| | | | | 1 and state 3 |
| | | | | indicates level |
| | | | | of occlusion. |
| | | | | If occlusion |
| | | | | increases, |
| | | | | alert doctor. |

### Example 4

General Glycemia Management

Glycemia patterns in diabetes and pre-diabetes patients can be monitored. Glucose and/or oxygen sensors can be implanted in the patients. pH sensors may also be implanted in the patients. In addition, the patients can be wearing one or more additional monitors of temperature, heat flux, motion (such as by IMU), heart rate, sweat rate, geolocation, location beacon (e.g. iBeacon), and breathing. Information may also be obtained from the user regarding what they eat, food photos, food diary, etc. Motion and quality of motion, skin temperature and heat flux, sweat, breathing and specific patent locations may be measured (e.g., using one or more monitors). Combining the data from the glucose sensor and oxygen sensor with the data from the additional monitors can be used to the underlying dynamics of particular glycemic (variability; hyper; or hypo) events.

For example, measuring glucose levels and oxygen levels and combining glucose and oxygen data with motion data and/or heat flux data can help determine whether glycogen is being dumped from muscle which helps to dampen subsequent hyperglycemic events.

In a further example, data from the glucose sensor and oxygen sensor may be combined with data measuring heart rate, sweat, and/or motion; an increase in heart rate and/or sweat combined with motion from the patient may not indicate a problem, whereas an increase in heart rate and/or sweat combined with the absence of motion from the patient may indicate current or imminent hypoglycemia.

For example, high levels of glucose and low pH detected by the glucose sensor and pH sensor may indicate ketoacidosis. This data combined with data regarding activity level and/or breathing rate may indicate that a signal needs to be provided to the user and/or caregiver. On the other hand, low glucose and low pH with high activity may indicate ketosis due to fat metabolism.

For example, data from the glucose sensor and oxygen sensor may be combined with data from geolocation/iBeacons and can provide data indicating if the patient went to the gym or a fast food restaurant, for instance. Analysis of 2 hour post-prandial glucose levels can further strengthen the conclusion that high glycemic index food was ingested. This data, in combination with glucose profiles, can point to behaviors that the patient may benefit from coaching.

Depth and duration of exercise modulates glycemic variability, mean amplitude of glycemic excursions (MAGE), and 2 hour postprandial (PP). Correlating motion/exercise to MAGE enables health-related feedback to be offered to users, such as coaching and optimization of exercise programs for prediabetics.

### Example 5

Monitoring sleep apnea, asthma, chronic lung diseases, ARDS, and other respiratory distress

Users with sleep apnea, asthma, chronic lung diseases, ARDS, and/or respiratory distress may be monitored using methods described herein. Oxygen sensors, carbon dioxide sensors, and/or pH sensors may be implanted in the user. In addition, the user may be wearing a breathing rate/respiration rate monitor. By measuring the oxygen levels, carbon dioxide levels, pH, and/or breathing rate, it can be determined that the user may need to increase their oxygen levels, have their medication amounts altered, or see a doctor. A signal can be sent to the user recommending that they review their amount of oxygen, medicine, or to see a doctor.

### Example 6

Monitoring Infection or Progression Toward Chronic Wound

Lactate, pH, and/or oxygen sensors may be implanted in a user. The user may also be wearing a temperature monitor. The combination of data from the oxygen, pH and/or lactate sensors, combined with data from the temperature monitor, may allow monitoring of infection or progression toward chronic wound. For example, if the lactate and/or oxygen sensors were implanted in the foot of a patient with PAD or a wound in the foot, monitoring these levels may provide data that can be used to signal that care for the foot is needed or that the user should see a doctor.

### Example 7

Monitoring Amount of Anesthesia Being Administered During Surgery

Oxygen sensors may be implanted in a user prior to, or during, surgery. The user may also be wearing a heart rate monitor. The combination of data from the oxygen sensor, combined with data from the heart rate monitor, may allow monitoring of the amount of anesthesia being administered during surgery.

### Example 8

Evaluating Limb Viability, Surgical Flap Reperfusion, Limb Reattachment, Organ Transplant, or Cardiovascular Surgery

Oxygen, pH, and/or lactate sensors may be implanted in a user near the site of a surgical intervention such as surgical flap reconstruction, limb reattachment, organ transplant, or cardiovascular surgery. The user may also be wearing an optical-sensing wearable that samples tissue in or around the sensor. The user may also be wearing a monitor to sample systemic cardiovascular parameters (e.g. ECG, arterial blood oxygenation). The user may also be wearing a chest strap to monitor breathing rate, for example. The data from the oxygen, pH, and/or lactate sensor may be combined with data indicating blood pulse wave, blood volume fraction, microvascular saturation, electrical signals indicating heart rate, and the like. The combination of data may be used to monitor the response to physiological or anatomical structural therapy (e.g. surgical flap reconstruction, limb reattachment, organ transplant, or cardiovascular surgery). The combination of data may be used to assess recovery from trauma, hemorrhage, or REBOA. In general, changes in local tissue physiology may be indicator of local tissue response (or non-response) to therapy. Changes in systemic cardiovascular output may serve as a check on the total body response to therapy. Disconnect between the two may serve to identify regional perfusion limitations. This information may be used during surgery to guide interventions, or it may be used post-surgery to inform potential follow-up interventions.

### Example 9

Diagnosing Sepsis

Oxygen, pH, and/or lactate sensors may be implanted in a user. The user may also be wearing a heart rate, respiratory rate, temperature, and/or blood pressure monitor. The combination of data from the oxygen, pH, and/or lactate sensors, combined with data from the heart rate, respiratory rate, temperature, and/or blood pressure monitor may be used to indicate or predict sepsis and shock. This data may be used to identify a red flag to indicate whether a particular treatment is helping a patient. This may be used in a hospital or military setting.

### Example 10

Diet and Oxygen in the Gut

Oxygen sensors may be ingested and could provide a stream of data on oxygenation with the gut. This could be coupled with data such as frequency of bowel movements, medications taken, and/or other systemic health monitors (e.g., heart rate, blood pressure).

### Example 11

Brain Trauma/Brain Injury

Oxygen sensors may be implanted outside or inside the skull could be used with other parameters such as heart rate, local blood perfusion (e.g., laser Doppler), and/or blood pressure. These monitors could help predict or diagnose poor oxygenation and need for intervention.

### Example 12

Islet Cell Viability

Oxygen sensors may be implanted in immunoisolation bags in a user. The user may also be wearing a flow rate monitor. The data from the oxygen sensor, combined with data from the flow rate monitor may be used in assessing the viability of islet cells.

### Example 13

Diagnosis of Allergic Reaction/Anaphylaxis

Oxygen, lactate, glucose, carbon dioxide and/or pH sensors may be implanted in a user. The user may also be wearing an external monitor that monitors blood pressure and/or breathing rate, for example. The combination of analyte data from the sensor with data from the external monitor can provide an objective metric to assess the progression/severity of an anaphylactic reaction. For example, individuals with latex-fruit allergies to foods such as bell peppers, paprika, or banana may be constantly exposed to small amounts of allergen and have to make decisions about the severity of exposure about when to seek medical help or to administer therapy in the form of corticosteroids and/or anti-histamines. For individuals with this precondition, continuous measurement of tissue analytes may provide insight into the loss of homeostasis during the onset and progression of a reaction. This information may be provided to the person experiencing the allergic attack to help with the decision making, or may be provided to a partner or caregiver to help with the shared decision making.

### Example 14

Monitoring Exercise Physiology

Oxygen, lactate, glucose, carbon dioxide and/or pH sensors may be implanted in a user. The user may also be wearing an external monitor that tracks movement (e.g. one or more IMU's) and provides a quantitative assessment of total motion and activity intensity. The monitor may track biometrics, for example heart rate, heart rate variability, respiration rate, local blood perfusion, and the like. The data from the lactate sensors (and potentially other sensors), for example, combined with the data from the standard wearable may be used to provide objective real-time metrics of the local muscle fitness and/or inform optimal scheduling of work-out activities. The data from the oxygen sensors, for example, combined with the data from the monitor may be used to provide insight into training regime (aerobic vs. anaerobic) and could be used to provide health-related feedback to the user, such as to guide intensity/exertion level.

### Example 15

Monitoring stress levels; Physiological monitoring of mind-body interventions

Oxygen, lactate, glucose, carbon dioxide and/or pH sensors may be implanted in a user. The user may also be wearing an external monitor that samples optical signals, electrical skin conductance, ECG, for example to measure respiration rate, galvanic skin response, heart rate, or similar. The combination of data from the oxygen, lactate, glucose, carbon dioxide and/or pH sensors, combined with data from the external monitor may be used in monitoring stress levels. A user could use this information to track the effectiveness of mindfulness and/or meditative therapy or intervention on physiological metrics of stress. Data from the external monitor can be combined with tissue analytes to assess magnitude and perseverance of physiological changes with/without mind-body intervention. Fourier analysis of tissue oxygen can provide insight into vessel spasm/movement as metric of stress response. The combined sensor and monitor data can provide a real-time objective metric to monitor the synchronization between breathing rate and heart rate (i.e. respiratory sinus arrhythmia). This metric can provide feedback to a user seeking to mitigate anxiety or stress response through controlled breathing or similar.

What is claimed is:

1. A system, comprising:
an implantable sensor configured to produce a signal associated with a concentration of oxygen in a tissue of a user;
a reader configured to be disposed on a surface of the user's skin, detect the signal produced by the implantable sensor, and calculate a concentration of oxygen;
a monitor configured to produce a signal associated with at least one of a condition of the user or a condition of the user's environment; and
an analyzer communicatively coupled to the monitor and the reader, the analyzer configured to provide health-related feedback to the user based on the concentration of oxygen and the at least one of the condition of the user or the user's environment.

2. The system of claim 1, wherein:
the monitor is at least one monitor configured to produce a signal associated with altitude and activity level;
the analyzer is configured to provide exertion-related feedback to the user based on the concentration of oxygen, the altitude, and the activity level.

3. The system of claim 1, wherein:
the monitor is configured to produce a signal associated with a respiration rate of the user; and
the analyzer is configured to provide respiratory-distress related feedback to the user based on the concentration of oxygen and the respiration rate.

4. The system of claim 1, wherein:
the implantable sensor is configured to produce a signal associated with a local concentration of oxygen at a wound site;
the monitor is configured to produce a signal associated with temperature; and
the analyzer is configured to provide feedback on wound healing to the user based on the local concentration of oxygen at the wound site and the temperature.

5. The system of claim 1, wherein the implantable sensor is configured to be implanted inside the user's skull.

6. The system of claim 1, wherein the implantable sensor is configured to be implanted in the user's gut.

7. The system of claim 1, wherein the health-related feedback is exercise-related feedback.

8. A system, comprising:
an implantable sensor configured to produce a signal associated with a concentration of an analyte in a tissue of a user;
a monitor configured to produce a signal associated with a condition of the user; and
a reader configured to be disposed on a surface of the user's skin, the reader configured to:
detect the signal produced by the implantable sensor,
calculate a concentration of the analyte,
receive the signal from the monitor, and
provide health-related feedback to the user based on the concentration of the analyte and the condition of the user.

9. The system of claim 8, wherein:
the implantable sensor is at least one implantable sensor configured to measure a concentration of oxygen in tissue of the user and a concentration of carbon dioxide in tissue of the user;
the at least one implantable sensor is configured to emit a first optical signal associated with the concentration of oxygen;
the at least one implantable sensor is configured to emit a second optical signal associated with the concentration of carbon dioxide;
the monitor is configured to produce the signal associated with an activity level of the user;
the reader is at least one reader configured to be positioned on a surface of the user's skin above the at least one implantable sensor to receive the first optical signal and the second optical signal and calculate the concentration of the oxygen and the concentration of carbon dioxide; and
the reader is configured to provide feedback to the user associated with chronic obstructive pulmonary disease (COPD) based on the concentration of oxygen, the concentration of carbon dioxide, and the activity level.

10. The system of claim 8, wherein:
the implantable sensor is at least one implantable sensor configured to measure a concentration of oxygen in tissue of the user and a concentration of carbon dioxide in tissue of the user;
the at least one implantable sensor is configured to emit a first optical signal associated with the concentration of oxygen;
the at least one implantable sensor is configured to emit a second optical signal associated with the concentration of carbon dioxide;
the monitor is configured to produce the signal associated with an activity level of the user; and
the reader is at least one reader configured to be positioned on a surface of the user's skin above the at least one implantable sensor to receive the first optical signal and the second optical signal and calculate the concentration of the oxygen and the concentration of carbon dioxide; and
the reader includes a communication module configured to send a signal to a remote monitoring device such that the remote monitoring device can provide a caregiver with information related to the user's chronic obstructive pulmonary disease (COPD) based on the concentration of oxygen, the concentration of carbon dioxide, and the activity level.

11. The system of claim 8, wherein:
the implantable sensor is at least one implantable sensor configured to measure a concentration of glucose in tissue of the user and a pH of the tissue;
the at least one implantable sensor is configured to emit a first optical signal associated with the concentration of glucose;
the at least one implantable sensor is configured to emit a second optical signal associated with the pH;
the monitor is configured to produce the signal associated with an activity level of the user; and
the reader is at least one reader configured to be positioned on a surface of the user's skin above the at least one implantable sensor to receive the first optical signal and the second optical signal and calculate the concentration of the glucose and the pH; and
the reader is configured to provide feedback to the user associated with glycemic vents based on the concentration of glucose, the pH, and the activity level.

12. The system of claim 8, wherein:
the implantable sensor is at least one implantable sensor configured to measure a concentration of glucose in tissue of the user and a pH of the tissue;
the at least one implantable sensor is configured to emit a first optical signal associated with the concentration of glucose;
the at least one implantable sensor is configured to emit a second optical signal associated with the pH;
the monitor is configured to produce the signal associated with a location of the user; and
the reader is at least one reader configured to be positioned on a surface of the user's skin above the at least one implantable sensor to receive the first optical signal and the second optical signal and calculate the concentration of the glucose and the pH; and
the reader is configured to provide feedback to the user associated with glycemic vents based on the concentration of glucose, the pH, and the location of the user.

13. The system of claim 8, wherein:
the implantable sensor is configured to produce a signal associated with a local concentration of an analyte at a wound site;
the reader is configured to provide wound-healing feedback based on the local concentration of the analyte.

14. The system of claim 8, wherein:
the implantable sensor is configured to produce a signal associated with a local concentration of at least one of pH or lactate at a wound site;
the monitor is configured to produce the signal associated with a temperature; and
the reader is configured to provide at least one of infection or wound-healing feedback based on the local concentration of at least one of pH or lactate and temperature.

15. The system of claim 8, wherein
the implantable sensor is configured to produce a signal associated with a local concentration of the analyte at a wound site;
the monitor is configured to produce the signal associated with at least one of blood pulse wave, blood volume fraction, or microvascular saturation; and
the reader is configured to provide structural therapy response feedback associated with the wound site.

16. A system, comprising:
an implantable sensor configured to produce a signal associated with a local concentration of oxygen in a tissue of a user;
a monitor configured to produce a signal associated with at least one of a condition of the user or a condition of the user's environment; and
an analyzer configured to provide health-related feedback to the user based on the local concentration of oxygen and the at least one of the condition of the user or the condition of the user's environment.

17. The system of claim 16, wherein:
the implantable sensor is configured to be disposed in tissue of a lower extremity such that the local concentration of oxygen is a concentration of oxygen in the lower extremity;
the monitor is configured to produce the signal associated with a position of the lower extremity of the user;
the analyzer is configured to determine when the user moves between a laying position and a standing position based on the position of the lower extremity;
the analyzer is configured to a rate of change of oxygen concentration in the lower extremity when the patient moves between the laying position and the standing position; and
the analyzer is configured to provide circulation-related feedback to the user based on the rate of change of oxygen concentration in the lower extremity when the patient moves between the laying position and the standing position.

18. The system of claim 16, wherein:
the implantable sensor is configured to be implanted at a wound site; and
the analyzer is configured to provide wound-healing feedback to the user based on the local concentration of oxygen at the wound site.

19. The system of claim 16, wherein:
the monitor is configured to produce the signal associated with the user's heart rate; and
the analyzer is configured to provide anesthesia feedback during a medical procedure based on the local concentration of oxygen and the user's heart rate.

20. The system of claim 16, wherein:
the implantable sensor is configured to be implanted at a wound site;
the monitor is configured to produce the signal associated with systemic cardiovascular output; and
the analyzer is configured to identify atypical local perfusion based on the local concentration of oxygen at the wound site diverging from the systemic cardiovascular output.
